(19) United States
(12) Patent Application Publication
(10) Pub. No.: US 2010/0003311 A1
(43) Pub. Date: Jan. 7, 2010

(54) Antimicrobial Composition

(76) Inventor: Marco Mastrodonato, Milano (IT)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/304,204

(22) PCT Filed: Jun. 13, 2007
(86) PCT No.: PCT/GB2007/002197

§ 371 Date: Jul. 1, 2009

(30) Foreign Application Priority Data
Jun. 13, 2006 (IT) MI2006A001132

(51) Int. Cl. A01N 25/34; A01N 43/04; A01N 65/00; A61K 8/97; A61Q 11/00; A01P 1/00

(52) U.S. Cl. 424/443; 514/54; 424/725; 424/740; 424/734; 514/12; 424/58

(57) ABSTRACT

Use of a composition comprising hyaluronic acid or a pharmaceutically acceptable salt thereof, in the manufacture of a topical medicament for antimicrobial treatment of the skin or mucosa.

### FIELD OF THE INVENTION

This invention relates to antimicrobial compositions comprising hyaluronic acid or a salt thereof.

### BACKGROUND TO THE INVENTION

Hyaluronic acid, also referred to as hyaluronan or hyaluronate, is a non-sulphated glycosaminoglycan distributed widely throughout connective, epithelial and neural tissues. Hyaluronic acid is, under normal circumstances, a high molecular weight (HMW) glycosaminoglycan, which is ubiquitous in the extra-cellular matrix. It is produced mainly by fibroblasts and is involved in maintaining the water balance of tissues, in the distribution and transport of proteins and in maintaining an intact extra-cellular matrix structure. In sites of inflammation or tissue injury, HMW hyaluronic acid may be depolymerised, to form low molecular weight (LMW) fragments, by the activity of oxygen radicals or by enzyme activity by hyaluronidase, β-glucuronidase or hexosaminidase.

The prevention of microbial infection is a constant challenge to healthcare professionals. Any area of damage to the skin or a mucosal membrane of a human or animal can result in a microbial infection. Although a large number of anti-microbial compositions are known, there remains a clear need for the provision of effective anti-microbial compositions.

### SUMMARY OF THE INVENTION

The present invention is based on the surprising realisation that hyaluronic acid is effective as a topical anti-microbial agent.

According to a first aspect of the invention, a composition comprising hyaluronic acid or a pharmaceutically acceptable salt thereof is used in the manufacture of a topical medicament for antimicrobial treatment of the skin or mucosa.

According to a second aspect of the invention, a method of reducing the microbial load on the skin or mucosa comprises contacting the skin or mucosa with a composition comprising hyaluronic acid.

According to a third aspect of the invention, a composition comprises hyaluronic acid or a pharmaceutically acceptable salt thereof and one or more plant extracts with antimicrobial activity.

According to a fourth aspect of the invention, a woven material is coated or impregnated with a composition comprising hyaluronic acid or a pharmaceutically acceptable salt thereof and one or more plant extracts with antimicrobial activity.

### DESCRIPTION OF THE FIGURE

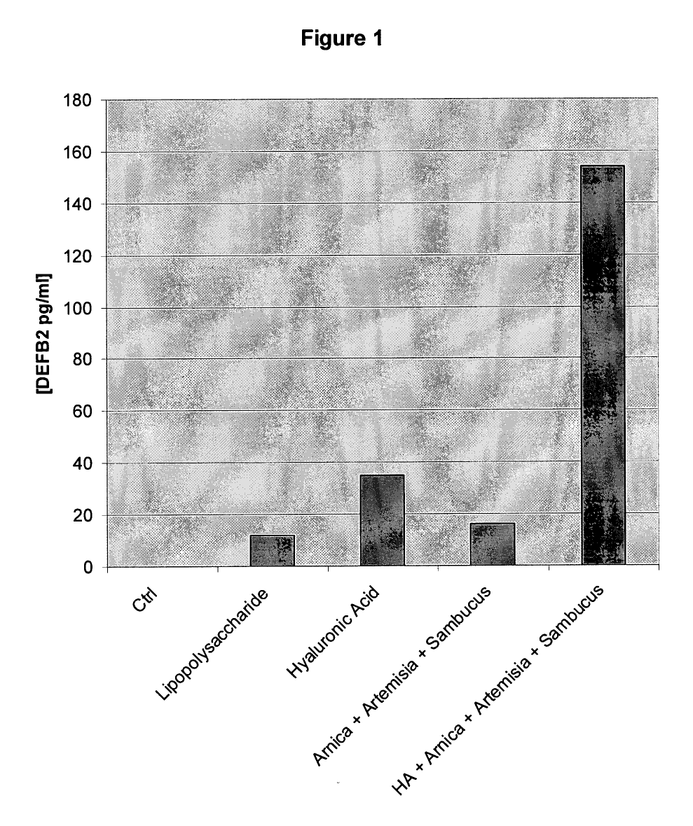

The invention is described with reference to the accompanying drawing, wherein, FIG. 1 is a graph illustrating the stimulation of β-defensin production in human keratinocytes by hyaluronic acid and plant extracts, and the synergy exhibited when both hyaluronic acid and plant extracts are used in a single composition.

### DETAILED DESCRIPTION OF THE INVENTION

The present invention is based on the finding that hyaluronic acid is useful as a topical anti-microbial agent at sites of tissue damage.

As used herein, the term “hyaluronic acid” is to be given its usual meaning in the art. Alternative names for hyaluronic acid include hyaluronan and hyaluronate. For the avoidance of doubt, hyaluronic acid is a polymer of disaccharides, each disaccharide consisting of D-glucuronic and D-n-acetyl glucosamine, linked via alternating β-1,4 and β-1,3 glycosidic bonds. Hyaluronic acid can be many thousand dissacharide repeats in length, with polymers ranging from 5 thousand to 20 million Da in vivo. According to the present invention, any sized hyaluronic acid may be used; the term “hyaluronic acid” includes both high molecular weight hyaluronic acid and low molecular weight hyaluronic acid. These terms are well known in the art; for the avoidance of doubt, low molecular weight hyaluronic acid has a molecular weight of less than 400 kDa, more preferably less than 100 kDa, yet more preferably less than 20 kDa, for example between 1 and 10 kDa. High molecular weight hyaluronic acid has a molecular weight greater than 400 kDa, more preferably greater than 800 kDa.

Hyaluronic acid or a salt of hyaluronic acid can be used according to the present invention. Preferably, the salt is a pharmaceutically acceptable salt. Examples of pharmaceutically acceptable salts are alkali metal salts such as sodium or potassium salt or alkaline earth metal salts such as magnesium or calcium salt.

The compositions of the invention are useful in stimulating an antimicrobial response when applied topically to the skin or mucosa. As used herein, the term “topically” refers to administration of the composition to a specific region of the skin or mucosa; the term “topically” is recognised in the art. Topical administration occurs at the localised region where the antimicrobial action of a composition according to the invention is required. Preferably, the composition is applied topically to a region of the skin or mucosa that has or is suspected of being affected by a microbial infection or other disorder caused by the detrimental presence of microbes. In a preferred embodiment, the infection originates at a site of damage to the skin or mucosa, such that the skin or mucosa is broken. A preferred site of infection is a lesion or wound i.e. an area where the skin or mucosa is burnt, torn, cut or punctured. In this embodiment, a composition according to the invention can be applied directly to the lesion or wound and/or the surrounding area.

The compositions of the invention are useful in treating any disorder of the skin or mucosa that is caused by or results in the detrimental presence of one or more microbes. Contacting the skin or mucosa with a composition according to the invention will reduce the microbial load, i.e. the number of microbes in that area of contact. Therefore, a method of treating a disorder of the skin or mucosa that is caused by or results in the detrimental presence of one or more microbes involves contacting the skin or mucosa with a composition of the invention, thereby reducing the number of microbes present and treating the disorder.

A large number of skin and mucosal disorders are known to be caused by microbes. A preferred skin or mucosal disorder that can be treated and/or prevented using a composition of the present invention is an ulcer, i.e. an open sore of the skin or mucosa that is usually caused by an initial abrasion followed by microbial infection. An ulcer at any stage, from 1 to 6 in the Merck Manual classification system, which is known in the art, can be treated within the scope of the invention. Ulcers are more prevalent in patients with diabetes; treatment of diabetic ulcers, in particular diabetic foot ulcers, is therefore a preferred embodiment. Ulceration or the oral mucosa can also be treated using the compositions of the present invention.

A preferred skin disorder that can be treated and/or prevented using a composition of the present invention is acne. As used herein, the term “acne” refers to any skin condition comprising a blocked pore of a pilosebaceous unit. The term “acne” includes the presence of white heads (closed comedones), blackheads (open comedones), papules, pustules, nodules and cysts. The term “acne” includes non-inflammatory acne, such as minor blackheads and whiteheads, and inflammatory acne wherein an immune response causes the blocked follicle to become inflamed, causing pustules, nodules or cysts. Preferably, the acne is characterised by the presence of Propionibacterium acnes.

In a preferred embodiment, the acne is Acne vulgaris. All forms of Acne vulgaris, from mild through moderate to severe, are within the scope of the invention. The severe forms of Acne vulgaris are sometimes referred to as Ance congloblata and Acne fulminans. Acne congloblata is the most severe form of acne vulgaris and is characterised by numerous lesions which are often connected. Acne fulminans is an acute onset version of Acne congloblata. The term “acne” also includes folliculitis, in particular gram-negative folliculitis, and pyoderma faciale, a type of facial acne that affects only females.

Psoriasis can be treated using a composition of the present invention. The exact cause of psoriasis is unknown, but it is known that microbial infections, in particular streptococcal infections, can trigger a psoriatic event. As used herein, the term “psoriasis” is to be given its usual meaning in the art, i.e. a chronic skin disease characterised by red patches covered with white scales. The composition of the invention is used preferably to treat guttate psoriasis.

The compositions of the present invention are useful in preventing and treating microbial infection. As used herein, the term “microbial infection” refers to the detrimental colonisation of a host organism by one or more microbial species. The host organism can be animal or human and is preferably human. The microbe causing the infection is preferably a bacteria, yeast, virus, or fungus. More preferably, the microbial infection is a bacterial infection.

The term “skin” is to be given its normal meaning in the art, i.e. the external organ comprising epithelial tissue. The word “mucosa” is well known in the art and comprises all mucous barriers in the body, e.g. gastro-intestinal, pulmonary, sublingual, buccal, rectal, vaginal, nasal, urethral and ocular barriers.

The inventor has surprisingly found that hyaluronic acid can induce expression of defensins and other innate immunity antibiotic peptides. The stimulation of β-defensin 2 by hyaluronic acid has been observed by the patent inventor. Without wishing to be bound by theory, it is thought that the surprising discovery that hyaluronic acid has antimicrobial activity is due to its ability to induce expression of innate immunity antibiotic peptides such as β-defensin 2, a cysteine rich low molecular weight peptide produced by epithelial cells which exhibits antimicrobial activity. It has been found by the present inventor that hyaluronic acid is able to determine the release of β-defensin 2 by epithelial surfaces (both mucosa and skin); hyaluronic acid therefore stimulates β-defensin 2 production. β-defensin 2 is a molecule of the “defensin” family, peptides of approximately 3 KDa with proven broad spectrum antibacterial and antimicrobial action; the UniProt accession number for human β-defensin 2 is O15263. Low molecular weight hyaluronic acid is thought to be most effective at stimulating production of innate immunity antibiotic peptides such as β-defensin 2. The application of hyaluronic acid to the skin is thought to increase the amount of innate immunity antibiotic peptides such as β-defensin 2 that are present and therefore provide an increased level of innate immunity. The stimulation of defensin and other innate immunity antibiotic peptide expression in sites of skin and mucosa injury may therefore facilitate tissue repair by providing innate immunity and decreasing the possible bacterial contamination and colonisation of tissues by opportunistic pathogens.

In one embodiment, the composition according to the invention, that is useful as an anti-microbial agent, contains hyaluronic acid as the only active anti-microbial agent. However, one or more other anti-microbial agents can be included in the composition. A preferred additional anti-microbial agent is an antibiotic. Any antibiotic may be used; the antibiotic that is most suitable for the required use will be apparent to the skilled person. Preferably, antibiotics are selected from the group consisting of clindamycin, erythromycin, benzylpenicillin, tetracycline, chloramphenicol, vancomycin and linezolid.

One or more anti-inflammatory agents may also be included in a composition of the invention. Steroidal anti-inflammatory agents, such as cortisone, or non-steroidal anti-inflammatories (NSAIDS), such as aspirin and ibuprofen (that inhibit cyclooxygenase isoenzymes) are within the scope of the invention.

A number of plant extracts are known to have antimicrobial and/or anti-inflammatory activities. In a preferred embodiment of the invention, a composition comprises hyaluronic acid and one or more plant extracts. Preferably, the plant extracts have an antimicrobial and/or anti-inflammatory effect themselves, which improves synergistically when combined with hyaluronic acid.

In a preferred embodiment, the composition comprises, in addition to hyaluronic acid, one or more plant extracts selected from Arnica Montana, Sambucus nigra and Artemisia vulgaris. In a further preferred embodiment, the composition additionally comprises an extract of Piper betel. Preferably, the composition comprises hyaluronic acid, Arnica Montana extract, Sambucus nigra extract and Artemisia vulgaris extract.

Each of the Arnica montana (Wolf's bane), Sambucus nigra (elderberry), Artemisia vulgaris (sage brush) and Piper betel extracts are known individually for their anti-microbial action. However, it has now been found that combining Arnica and/or Sambucus and/or Artemisia, and optionally Piper betel, with hyaluronic acid provides a surprising synergy and show a greater anti-microbial effect than that obtained from the sum of the effects obtained after the separate administration of each of the components. The synergy is clearly demonstrated by FIG. 1.

FIG. 1 illustrates that hyaluronic acid stimulates β-defensin production by human keratinocytes significantly, and considerably more than lipopolysaccharide, which is known to induce β-defensin 2 expression. FIG. 1 also illustrates the considerable synergy that is observed when a combination of hyaluronic acid, Arnica extract, Sambucus extract and Artemisia extract is used. The data was obtained by measuring β-defensin production in culture medium containing human keratinocytes when contacted with the various compositions.

The active ingredients in the Arnica extract are contained in the rhizome and in the flower heads, which contain an essential oil that contains tri-terpene alcohols (arnicin, arnidiol and faradiol), sesquiterepene lactones, isoquercetin, astragalin, luteolin-7-glucoside, flavonoids, organic acids (phenolic acids, oleic acids, lauric acid and palmitic acid, and thymol). Arnica extracts are known in the art and can be routinely prepared by the skilled person. Preferably, the extract is the dry aqueous extract with CAS No. 68990-11-4.

The active ingredients in Sambucus nigra are found throughout the whole plant, but well-matured flowers are most commonly used to obtain extract as these are rich in flavonoids (rutin and isoquercetin), essential oils, tri-terpenes, tannins, Sambucus-nigrine, cyanogenetic glucoside, sambucine alkaloid, organic acids (valeric and malic), mucilage, resins, antiocianus and coline. Sambucus nigra extracts are known in the art and can be routinely prepared by the skilled person. Preferably, the extract is the dry aqueous extract with CAS No. 8477545-1.

Artemisia extract is largely taken from the flowers and contains the active ingredients as an essential oil containing cineole, thujone, linaloloe borneolo, a sesquiterpene lactone (vulgarine), rutin, curarine, tannins and resins. Artemisia extracts are known in the art and can be routinely prepared by the skilled person. Preferably, the extract is the dry hydroalcoholic extract with CAS No. 84603-58-7.

Piper betel is a climbing shrub native of South East Asia. The actively ingredients of the extract comprise an oil containing phenols (chavibetol and chavicol) and a bi-cyclical sesquiterpene, cadinene. The Piper betel extract can be routinely prepared by the skilled person. Preferably, the extract is the oil extract with CAS No. 84775-81-5.

For the avoidance of doubt, all combinations of each of the composition ingredients described herein are within the scope of the invention. Compositions comprising hyaluronic acid alone and in combination with Arnica extract, and/or Sambucus extract, and/or Artemisia extract are within the scope of the invention. Preferred combinations are hyaluronic acid and Arnica extract; hyaluronic acid, Arnica extract, Sambucus extract and Artemisia extract; hyaluronic acid, Arnica extract and Sambucus extract; hyaluronic acid, arnica extract and Artemisia extract; hyaluronic acid, sambucus extract and Artemisia extract. Piper betel extract may be included in any of these compositions.

The compositions for use in the present invention should be suitable for topical application to the skin and consist of the claimed compound in a pharmaceutically acceptable cream, ointment or gel. The composition can be hydrophillic or hydrophobic. The composition can be an aqueous composition, although other suitable solvents, such as alcohols or other organic solvents, may be used. A combination of solvents may also be used.

In addition to the antimicrobial ingredients, one or more pharmaceutical excipients, which do not have antimicrobial activity, may be included in a composition of the invention. Examples of suitable excipents include diluents, glidants, preservatives, gums, sweeteners, coatings, binders, disintegrants, lubricants, suspending agents, solvents, dispersants, colourants, flavourings, anti-adherence agents, surfactants, plasticizers, emulsifiers, chelating agents and emollients. A preferred excipient is an emollient.

A suitable cream may be prepared by incorporating the active compound in a topical vehicle such as light liquid paraffin, dispersed in a aqueous medium using surfactants. An ointment may be prepared by mixing the active compound with a topical vehicle such as a mineral oil or wax. A gel may be prepared by mixing the active compound with a topical vehicle comprising a gelling agent.

Topically administrable compositions may also comprise a matrix in which a pharmaceutically active compound of the present invention is dispersed so that the compound is held in contact with the skin in order to administer the compounds transdermally.

A composition of the invention is preferably a cream, salve, ointment, gum, toothpaste, mouthwash or shampoo. The preferred formulation will be apparent to the skilled person and will vary based on the required use. For example, to treat a mouth ulcer, a simple gum may be suitable, whereas to treat acne, a cream, salve, ointment, lotion or facewash may be preferable. The composition can be coated onto, or impregnated onto, woven or non-woven materials that are intended to contact the skin or mucosa topically. A preferred woven material that can be coated or impregnated with a composition of the invention is a wound dressing such as a sticking plaster or bandage. To treat acne, a woven face wipe impregnated with a composition of the invention may be suitable.

Each of the agents in the composition can be used at any suitable amount that would be apparent to the skilled person. In a preferred embodiment, each agent (hyaluronic acid and each of the plant extracts) is present between 0.1% w/w and 5% w/w, for example 0.1% w/w to 2% w/w, 0.2% w/w to 1% w/w, for example 0.5% w/w.

In a further embodiment, the composition according to the invention additionally comprises lactoferrin. Lactoferrin is a glycoprotein with anti-microbial activity that is part of the innate immune response, mainly at mucosal surfaces. As used herein, the term “lactoferrin” is to be given its usual meaning in the art. For the avoidance of doubt, any human or animal lactoferrin may be used according to the invention. An example of a suitable lactoferrin is human lactoferrin, which has a molecular weight of 77.05 kDa and the UniProt accession number P02787.

Suitable compositions of the invention are disclosed in the following examples. All % are % w/w.

It should be noted that, in each of these examples, the lactoferrin and/or Piper betel components can be removed and the balance made up using additional distilled water.

### EXAMPLE 1

### Cream for Topical Use

| | |
|---|---|
| (Lactoferrin | 0.50%) |
| Sodium hyaluronate | 0.50% |
| Arnica montana | 0.50% |
| Artemisia vulgaris | 0.50% |
| Sambucus nigra | 0.50% |
| (Piper betel | 0.50%) |
| Ethylhexyl palmitate | 10.00% |
| Cetearil alcohol - PEG-20 Stearate | 9.00% |
| Olus | 5.00% |
| Cetearil alcohol | 2.00% |
| Glycerine | 2.00% |
| Dimethicone | 1.00% |
| Fenossietanol | 0.70% |
| Imidazolidinil Urea | 0.30% |
| Propylparaben | 0.15% |
| Methylparaben | 0.15% |
| Disodium EDTA | 0.10% |
| Distilled water | 66.60% |
| Total | 100.00% |

### EXAMPLE 2

### Mouthwash

| | |
|---|---|
| (Lactoferrin | 0.50%) |
| Sodium hyaluronate | 0.50% |
| Arnica montana | 0.50% |
| Artemisia vulgaris | 0.50% |
| Sambucus nigra | 0.50% |
| (Piper betel | 0.50%) |
| Potassium sorbate | 0.30% |
| Benzoate sodium | 0.30% |
| Hydroxyethyl cellulose | 0.30% |
| Disodium EDTA | 0.10% |
| Benzalkonium chloride | 0.10% |
| PEG-40 Hydrogenated Castor Oil | 0.10% |
| Sodium saccharin | 0.05% |
| Flavouring | 0.03% |
| Distilled water | 95.72% |
| Total | 100.00% |

### EXAMPLE 3

### Toothpaste

| | |
|---|---|
| (Lactoferrin | 0.50%) |
| Sodium hyaluronate | 0.50% |
| Arnica montana | 0.50% |
| Artemisia vulgaris | 0.50% |
| Sambucus nigra | 0.50% |
| (Piper betel | 0.50%) |
| Sorbitol | 50.00% |
| Hydrate silica | 8.00% |
| Scarcosine sodium lauroil | 3.50% |
| Carbossimethil cellulose | 1.50% |
| Potassium sorbate | 0.30% |
| Benzoate sodium | 0.30% |
| PEG-40 Hydrogenated Castor Oil | 0.16% |
| Sodium saccharin | 0.10% |
| Flavouring | 0.50% |
| Distilled water | 32.64% |
| Total | 100.00% |

### EXAMPLE 4

### Vaginal Cream

| | |
|---|---|
| (Lactoferrin | 0.50%) |
| Sodium hyaluronate | 0.50% |
| Arnica montana | 0.50% |
| Artemisia vulgaris | 0.50% |
| Sambucus nigra | 0.50% |
| (Piper betel | 0.50%) |
| Liquid paraffin | 10.00% |
| Cetearil Alcohol - PEG-20 Stearate | 9.00% |
| Olus | 5.00% |
| Cetearil Alcol | 2.00% |
| Glycerine | 2.00% |
| Dimethicone | 1.00% |
| Fenossietanol | 0.70% |
| Imidazolidinil Urea | 0.30% |
| Propylparaben | 0.15% |
| Methylparaben | 0.15% |
| Disodium EDTA | 0.10% |
| Lactic acid | 0.10% |
| Distilled water | 66.50% |
| Total | 100.00% |

1. A method of reducing the microbial load on the skin or mucosa, comprising contacting the skin or mucosa with a composition comprising hyaluronic acid or a pharmaceutically acceptable salt thereof.

2. The method, according to claim 1, wherein the composition is applied topically at a site of damage to the skin or mucosa.

3. The method, according to claim 1, wherein the composition stimulates β-defensin 2 production.

4. The method according to claim 1, wherein the composition additionally comprises one or more plant extracts with antimicrobial activity.

5. The method, according to claim 4, wherein the one or more plant extracts are selected from Arnica montana, Sambucus nigra and Artemisia vulgaris.

6. The method, according to claim 5, wherein each of the Arnica montana, Sambucus nigra and Artemisia vulgaris extracts is present in the composition.

7. The method, according to claim 5, wherein the composition additionally comprises an extract of Piper betel.

8. The method, according to claim 1, wherein the composition additionally comprises lactoferrin.

9. The method, according to claim 1, wherein each ingredient is present in an amount from 0.1% to 2% w/w.

10. The method according to claim 1, which is antibacterial.

11. (canceled)

12. A composition comprising hyaluronic acid or a pharmaceutically acceptable salt thereof and one or more plant extracts with antimicrobial activity.

13. The composition according to claim 12, wherein the one or more plant extracts is, or are, selected from Arnica montana, Sambucus nigra, and Artemisia vulgaris.

14. The composition according to claim 13, wherein each of the Arnica montana, Sambucus nigra and Artemisia vulgaris extracts is present.

15. The composition according to claim 13 additionally comprising an extract of Piper betel.

16. The composition according to claim 12, wherein each ingredient is present in an amount between 0.1% and 2% w/w.

17. The composition according to claim 12, which has been formulated for topical use.

18. The composition according to claim 17, in the form of a cream, ointment, salve, gum, mouthwash, shampoo or toothpaste.

19. A woven material coated or impregnated with a composition comprising hyaluronic acid or a pharmaceutically acceptable salt thereof and one or more plant extracts with antimicrobial activity.
